(19) United States
(12) Patent Application Publication
(10) Pub. No.: US 2014/0228407 A1
(43) Pub. Date: Aug. 14, 2014

(54) TETRA-PYRIDINE COMPOUNDS AND COMPOSITION FOR PROTECTING CELLS, TISSUES AND ORGANS AGAINST ISCHEMIA-REPERFUSION INJURY

(71) Applicants: Jerachmiel Y. Appelbaum, Jerusalem (IL); Edit Tshuva Goldberg, Rehovot (IL)

(72) Inventors: Jerachmiel Y. Appelbaum, Jerusalem (IL); Edit Tshuva Goldberg, Rehovot (IL)

(73) Assignees: YISSUM RESEARCH DEVELOPMENT COMPANY OF THE HEBREW UNIVERSITY, LTD., Jerusalem (IL); ATHENA CARDIONET LTD., Jerusalem (IL)

(21) Appl. No.: 14/130,065

(22) PCT Filed: Jun. 27, 2012
(86) PCT No.: PCT/US2012/044376

§ 371 Date: May 5, 2014

Related U.S. Application Data
(60) Provisional application No. 61/503,151, filed on Jun. 30, 2011.

(51) Int. Cl. C07D 213/36

(52) U.S. Cl. CPC C07D 213/36
USPC 514/332; 546/264; 435/1.1

(57) ABSTRACT

The invention provides a novel tetra-pyridine compound and a composition containing this compound for protecting and preserving cells, tissues and organs against ischemia-reperfusion injury. A method and use of the tetra-pyridine compound for preventing or treating ischemia-reperfusion injury or for protecting and preserving excorporeal tissue or organ from ischemia and oxygen radical-related damage are also provided.

### BACKGROUND OF THE INVENTION

1. Field of the Invention

The present invention relates to metal chelating compounds and compositions for protecting organs, tissue and cells from injury caused by ischemia-reperfusion.

2. Description of the Related Art

Ischemic injury results from a tissue restriction in blood supply leading to a mismatch between oxygen supply and demand, and may result in cell necrosis. However, restoration of oxygenated blood to an ischemic tissue, i.e., reoxygenation, give rise to a more severe tissue damage which is usually associated with programmed cell death, apoptosis. The link between these two events is well known as ischemia-reperfusion injury (IRI). Hence minimizing IRI has broad range clinical implications. IRI is regarded as a redox active metal and free radical mediated phenomenon that occurs during acute myocardial infarction, stroke, thrombolysis, and other pathological situations associated with ischemia followed by reoxygenation/reperfusion. This type of injury/damage to tissue and organs also occurs in the post-ischemic reperfusion during medical procedures such as cardiopulmonary bypass, percutaneous coronary intervention, coronary angioplasty and other thrombolytic procedures.

In early experiments, transition metals, including copper and iron, were found to play an essential mediatory role in reperfusion-induced myocardial damage. While copper, iron and zinc are the most abundant transition metals relevant to normal biological function and to sustaining life, these biologically important transition metals are also redox active metals. Hence, their intracellular transport is tightly regulated.

Under normal physiological conditions, these redox active metals are stored in situ within protective proteins like feritine and ceruloplasmin. However, under altered physiological situations as ischemia, they are frequently released from their intracellular stores and subjected to high oxygen level upon initiating reperfusion. The result is the triggering of redox-active catalysis processes and formation of harmful free radicals, so called reactive oxygen species (ROS) via the Fenton and Haber-Weiss reactions.

There is common agreement among the scientific community that ROS is involved in and mediates many diseases, syndromes and pathologies, such as heart and brain stroke, brain trauma, organ transplants rejection, various neurodegenerative diseases, arthritis, etc.

A similar problem with ischemia-reperfusion injury/damage exists with regard to organ preservation for transport of the organ for transplantation and for the transplant procedures. The growing need and the development of successful organ transplantation procedures throughout the world has raised the demand for methods of safe and prolonged storage of donor organs that eliminate or minimize any damage to the organ upon its transport, transplantation and subsequent reoxygenation.

The observation of oxygen-induced myocardial damage during reperfusion led to the concept of using oxygen free radical scavengers. However, rather than attempting to scavenge these short-lived and harmful free-radical species, U.S. Pat. No. 5,082,851 discloses a pharmaceutical composition containing a metal chelating agent, N,N,N′,N′-tetrakis-(2-pyridylmethyl)-ethylenediamine, as the active ingredient for protecting the heart, as well as excorporeal organs and cells, from ischemia-reperfusion damage. This high specificity metal chelating agent is thought to work by changing the redox potential of redox-active metals into an inactive form before the onset of reoxygenation and the resultant ROS generation.

Citation of any document herein is not intended as an admission that such document is pertinent prior art, or considered material to the patentability of any claim of the present application. Any statement as to content or a date of any document is based on the information available to applicant at the time of filing and does not constitute an admission as to the correctness of such a statement.

### SUMMARY OF THE INVENTION

The present invention relates to a tetra-pyridine compound of the formula

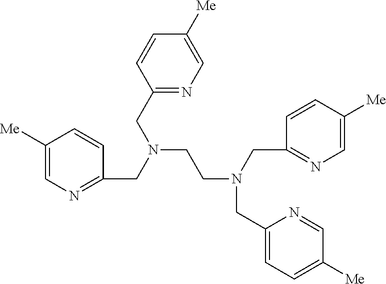 [SMILES: Cc1ccc(CN(CCN(Cc2ccc(C)cn2)Cc2ccc(C)cn2)Cc2ccc(C)cn2)nc1]

hereinbelow referred to as Compound #6.

The present invention also provides a composition that includes the above tetra-pyridine compound referred to as Compound #6 and a pharmaceutically acceptable excipient or carrier.

The present invention further provides an ischemia-reperfusion injury protecting composition and an organ preservation composition.

The present invention thus provides a novel and improved pharmaceutical composition containing a metal chelating agent aimed at better treatment or prevention of ischemia-reperfusion injury.

Another aspect of the present invention relates to a method for preventing or treating ischemia-reperfusion injury in which an effective amount of the above tetra-pyridine compound is administered to a patient at risk of ischemia-reperfusion injury.

A further aspect of the present invention relates to a method for protecting and preserving excorporeal tissues and organs by immersing an excorporeal tissue or organ in an excorporeal tissue and organ protecting and preserving effective amount of the above tetra-pyridine compound.

A still further aspect of the present invention relates to a method for protecting cells from ischemia and oxygen radical-related damage, comprising immersing cells in a solution which includes an ischemia and oxygen radical-related damage inhibiting effective amount of the above tetra-pyridine compound.

### BRIEF DESCRIPTION OF THE DRAWINGS

FIG. 1A is a graph along with raw data showing the results of an in vitro assay of hypoxia induction with N₂ (100%) and O₂ (less than 1%) in neonatal rat heart myocytes. LDH release is measured as a determination of cell apoptosis. The effect of various concentrations (30, 100 and 300 nm) of Compound #4 or Compound #6 was compared with 1 μM TPEN as a reference compound. FIG. 1B is a graph showing the effect of Compound #6 on propidium iodide (PI) staining following 3.5 hr. reoxygenation.

FIG. 2 is a graph along with raw data showing the results of cell death from ischemic insult in PC12 cells as determined by LDH measurement 2 hr. 50 min. of ischemia.

FIGS. 3A and 3B are graphs and raw data showing the results of the protective effect of Compound #4, Compound #6 and TPEN at various concentrations on cell death from ischemic insult in PC12 cells, as determined by LDH measurement.

FIG. 4 is a graph along with raw data showing the results of the protective effect of 1 μM Compound #6 compared to various concentrations of TPEN on cell death from ischemic insult in PC12 cells, as determined by LDH measurement.

FIG. 5 is a graph along with raw data showing the results of the protective effect of 1 μM and 3 μM Compound #6 compared to 1 μM, 3 μM and 5 μM of TPEN on cell death from ischemic insult in PC12 cells, as determined by LDH measurement.

FIGS. 6A and 6B are graphs showing the results of a dose-response function along with raw data (FIG. 6A) and protection percentile (FIG. 6B) at selected concentrations of compound #6 and TPEN.

FIG. 7 is a graph showing infarct size in a rat model of ischemia-reperfusion as a percentage of the Area At Risk (AAR) and as a percentage of the left ventricle (LV), and showing the AAR as a percentage of the LV.

### DETAILED DESCRIPTION OF THE INVENTION

The present inventors have synthesized a novel tetra-pyridine compound of the formula

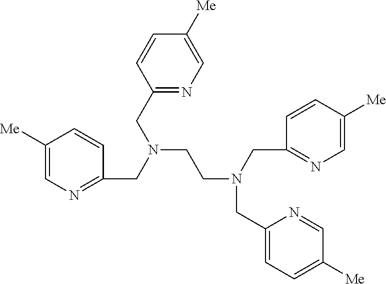 [SMILES: Cc1ccc(CN(CCN(Cc2ccc(C)cn2)Cc2ccc(C)cn2)Cc2ccc(C)cn2)nc1]

which is referred to as Compound #6 and is one aspect of the present invention. Another aspect of the present invention is a composition which includes the above tetra-pyridine compound (Compound #6) and a pharmaceutically acceptable excipient or carrier. Preferably, the composition according to the present invention is an ischemia and reperfusion injury protecting composition or a tissue or organ preserving composition. The composition can be administered to an individual who has experienced blood loss, has had a stroke or a cardiopulmonary arrest, is about to undergo or is undergoing a procedure such as surgery, or is an organ donor.

The tetra-pyridine compound and composition according to the present invention can be used in methods for preventing or treating ischemia-reperfusion injury (e.g., by reducing damage to tissue or organ following an ischemic event) and for protecting and preserving excorporeal tissues and organs (e.g., during storage and upon transplant into a recipient where reperfusion occurs). Thus, the methods of the present invention protect individuals (or tissues and organs transplanted into a recipient) from ischemic damage and reperfusion injury. The method for preventing or treating ischemia-reperfusion injury involves administering an effective amount of the tetra-pyridine compound (Compound #6), such as in the composition according to the present invention, to a patient at risk of ischemic-reperfusion injury. Administration of the effective amount to prevent or treat ischemia-reperfusion injury can be by any route discussed herein below, including bathing or immersing the tissue or organ of the patient in a composition of the present invention, while the patient is undergoing a medical/surgical procedure. This bathing or immersing of the entire tissue or organ can also be used for protecting and preserving an excorporeal tissue or organ for purposes of transplanting into a recipient. In a method for protecting and preserving an excorporeal tissue or organ, an alternative embodiment is to treat the tissue or organ prior to harvesting the tissue or organ from a donor, optionally in conjunction with bathing or immersing the entire tissue or organ in a liquid containing an excorporeal tissue and organ preserving effective amount of the tetra-pyridine compound of the present invention.

In particular, the methods according to the present invention have wide clinical significance in protecting the heart from ischemia-reperfusion injury during heart surgery (on-pump and off-pump), coronary interventions (balloon and stent), acute ischemic syndromes, arrhythmia management and organ transplantation. It may also find utility in assisting cardiologists to reduce ischemia-reperfusion injury followed by arrhythmias during angioplasty for coronary stent placement or valvular interventions. While cardiac patients are a particularly preferred population for the present methods, patients having other tissues or organs, such a kidneys, lungs, brain, etc., in need of medical or surgical intervention are included in the methods of the present invention. Representative individuals or patients include those who have had a stroke or are at risk of having a stroke and those who are undergoing surgery (e.g., neurosurgery)

The methods according to the present invention may also be useful for treating patients experiencing a trauma that may have resulted from injury in the battlefield or in an accident.

A further aspect of the present invention relates to a method for protecting cells from ischemia and oxygen radical-related damage. This method involves immersing cells in a solution which includes an ischemia and oxygen radical-related damage inhibiting effective amount of the tetra-pyridine compound of the present invention. The cells are preferably mammalian, most preferably human.

“Injury” can be broadly characterized as reversible and irreversible cell injury. For example, reversible cell injury can lead to heart dysfunction usually from arrhythmias and/or stunning. Stunning is normally characterized as loss of left-heart pump function during restoration of blood flow following periods of ischemia. If severe, it can lead to the death of the heart, usually from arrhythmias, even though the heart cells themselves are not initially dead. Irreversible injury by definition arises from actual cell death which may be fatal depending upon the extent of the injury. The amount of cell death can be measured as infarct size. Following by-pass cardiac surgery, during recovery from cardioplegic arrest, if the conditions are adequate, the heart can be restored substantially to normal function of the tissue by reperfusion, with minimal infarct size. The most common ways to assess return of function of a heart are by measuring pressures that the heart can generate; heart pump flow; and the electrical activity of the heart. This data is then compared to data measured from pre-arrest conditions. In the present specification, the terms “injury” and “damage” may be used interchangeably.

The term “organ” is used herein to include circulatory organs such as the blood vessels, heart, respiratory organs such as the lungs, urinary organs such as the kidneys or bladder, digestive organs such as the stomach, liver, pancreas or spleen, reproductive organs such as the scrotum, testis, ovaries or uterus, neurological organs such as the brain and spinal cord.

The term “tissue” is used herein in its broadest sense and refers to any part of the body exercising a specific function including organs and aggregates of cells that have similar morphology and function. Other examples include conduit vessels such as arteries or veins or circulatory organs such as the heart, tissues of organs like respiratory organs such as the lungs, urinary organs such as the kidneys or bladder, digestive organs such as the stomach, liver, pancreas or spleen, reproductive organs such as the scrotum, testis, ovaries or uterus, neurology organs such as the brain and spinal cord, skeletal tissues of muscles and bones, cartilage, collagen and fibrous tissues.

The term “cells” is used herein in its broadest sense and refers to any cell of the body exercising a specific function including germ cells such as spermatozoa or ovum and somatic cells such as skin cells, heart cells, (i.e., myocytes), pancreatic cells, nerve cells, kidney cells and isolated cells from various body organs. In addition the term “cells” used herein refers inter alia to all kind of stem cells including embryonic and fetal stem cells, adult stem cells or tissue-specific stem cells, cord blood and amniotic stem cells, embryonic stem cells, induced pluripotent stem cells and peripheral blood stem cells.

The body, individual or patient may be a human or an animal such as a livestock animal (e.g., sheep, cow or horse), laboratory test animal (e.g., mouse, rabbit or guinea pig) or a companion animal (e.g., dog or cat), particularly an animal of economic importance. Preferably, the body, individual or patient is human.

The composition according to the present invention, in which an ischemia and reperfusion injury protecting composition or an organ preservation composition are preferred embodiments, may be suitable for administration to the tissue or organ or the patient as a liquid formulation, for example, a solution, syrup or suspension, that is ready for use or alternatively may be administered as a dry product for constitution with water or other suitable vehicle before use. Alternatively, the composition may be formulated as a dry product, e.g., dry powder, for dissolution or suspension with water or other suitable vehicle prior to use. Any number of solvents, including preferably sterile water, can be used in the composition in liquid form or to dissolve or resuspend a dry powder composition that requires dissolution or resuspension prior to use. Liquid compositions may be prepared by conventional means with pharmaceutically acceptable additives such as suspending agents, emulsifying agents, non-aqueous vehicles, preservatives and energy sources. The composition can also be in tablet form as well as in the form of an aerosol which could be administered via oral, nasal and by transdermal routes as the composition alone or via any of suitable vehicles.

Besides including a pharmaceutically acceptable excipient or carrier as is common in the art, the composition according to the present invention, when stored for any amount of time as a liquid, may also include one or more stabilizers, such as organic sugars, sugar alcohols or saccharides, amino acids, and low molecular weight polypeptides or proteins (e.g., serum albumin) to prevent degradation of the active ingredient and to prolong the shelf life of a liquid composition.

The composition of the present invention may be administered intravenously or be administered both intravenously and intraperitoneally or directly accessing a major artery such as the femoral artery or aorta in patients who have no pulse from massive exsanguinations, or in the carotid artery or another artery during aortic dissection to protect the brain from hypoxia or ischemia. In one embodiment, the composition may be administered intravenously and intraperineally simultaneously, the perineum acting as, in effect, a reservoir of composition for the bloodstream as well as acting on organs in the vicinity with which it comes into contact. This is particularly suitable for a trauma victim, such as one suffering shock.

The composition can also be infused or administered as a bolus intravenous, intracoronary or any other suitable delivery route for protection during cardiac intervention such as open heart surgery (on-pump and off-pump), angioplasty (balloon and with stents or other vessel devices), any percutaneous coronary intervention (PCI) procedure, percutaneous valvular therapy, endovascular and carotid interventions. The composition can also be used for various stent coatings, including bifurcated stents, as part of biodegradable stent compositions, and as independent clot-busters, to protect and preserve cell viability. The composition also can be administered as a bolus, for example, by a first-responder (e.g., an armed services medic, an Emergency Medical Technician (EMT) or any other trained medical personnel) to an individual experiencing a major hemorrhagic event or a heart and brain stroke, traumatic brain injury (TBI) or cardiopulmonary arrest. Alternatively or in addition to a bolus administration, the composition as an ischemia and reperfusion injury protecting composition can be administered as a slow-drip or infusion over a period of time. For example, a slow-drip or infusion can be administered at the scene of trauma, during transport to a medical facility, and/or once the individual reaches a medical facility. Physiologically, the period immediately after injury or trauma is critical and is sometimes referred to as the “golden hour” but administration of an ischemia/reperfusion protection composition to an individual can be continued for up to 72 hours or longer (e.g., up to 1 hour, 2 hours, 4 hours, 6 hours, 8 hours, 12 hours, 18 hours, 24 hours, 36 hours, 48 hours, 60 hours, 90 hours, or more). As an alternative to a slow-drip or infusion, a bolus of the ischemia and reperfusion injury protecting composition can be administered multiple times over, for example, a 24, 48 or 72 hour period of time.

Generally, an individual who has experienced a major hemorrhagic event will receive a blood transfusion upon reaching a medical facility, which, depending upon the circumstances, may take only a few minutes following the injury or may take up to several hours or more. In some instances, the ischemia and reperfusion injury protecting composition can be administered to an individual as soon as a potential ischemia or reperfusion injury is recognized, which may be after a blood transfusion has already begun. Those of skill would realize that the ischemia and reperfusion injury protecting composition could be administered coincidentally with a blood transfusion or plasma replacement and, in some instances, the ischemia and reperfusion injury protecting composition can be combined directly with the blood or plasma and administered to an individual.

A tissue or organ at risk of ischemia-reperfusion injury may be contacted by delivering the composition intravenously to the tissue or organ. This involves using blood as a vehicle for delivery to the tissue or organ. Preferably, the administration is by injection via the blood stream heading for the ischemic tissue or organ. For example, in the case of acute myocardial infarction, the tetra-pyridine compound or the composition of the present invention may be injected intra-arterially, such as via a balloon catheter, immediately following removal of the clot that is causing the occlusion in order to enable reperfusion of the tissue or organ. In particular, the composition may be used for blood cardioplegia. Alternatively, the composition may be administered directly as a bolus by a puncture (e.g., by syringe) directly to the tissue or organ, which may be particularly useful when blood flow to a tissue or organ is limiting. While the composition may be administered directly to the tissue or organ or to exposed parts of the internal body to reduce ischemia-reperfusion injury, the composition may also be administered systemically to the individual/patient, such as by intravenous administration. Further means of administering the composition for protecting and preserving a tissue or organ may also include the oral, skin or nasal routes in the form of an aerosol, powder, solution or paste.

The composition according to the invention may be used as part of a cardioplegic solution, e.g., injected into the aortic root or the coronary Ostia to induce cardiac arrest during open heart surgery so as to protect against ischemia-reperfusion injury. Thus, for a surgical or diagnostic procedure, such a composition could be administered to provide localized arrest of the target tissue or organ as well as protection during reperfusion.

The composition may also be formulated as depot preparations. Such long acting formulations may be administered by implantation (e.g., subcutaneously or intramuscularly) or by intramuscular injection. Thus, for example, the composition according to the invention may be formulated with suitable polymeric or hydrophobic materials (e.g., as an emulsion in an acceptable oil or ion exchange resins, or as sparingly soluble derivatives, for example, as a sparingly soluble salt).

The composition may be delivered according to one of or a combination of the following delivery protocols: intermittent, continuous and one-shot. Accordingly, in another aspect of the present invention, the composition may be administered as a single dose of the composition.

In another aspect of the invention, the composition may be administered by intermittent administration. A suitable administration schedule is a 2 minute induction dose every 20 minutes throughout the arrest period. The actual time periods can be adjusted based on observations by one skilled in the art administering the composition, and the individual selected. The present invention also provides a method for intermittently administering a composition for reducing ischemia-reperfusion injury to the tissue or organ.

The composition can also be used in continuous infusion with both normal and injured cells, tissues or organs, such as the heart. Continuous infusion also includes static storage of the tissue or organ, whereby the tissue or organ is stored in a composition according to the invention. For example, the tissue or organ may be placed in a suitable container and immersed in a composition (or solution) for transporting donor organs or tissues from a donor to recipient.

Ischemic damage/reperfusion injury also can occur in organs intended for transplant. The organ donor can be in a persistent vegetative state, or can be alive and healthy and a suitable match for the recipient. In addition or alternatively to administering the ischemia-reperfusion injury protecting composition to an individual, one or more harvested organs, such as heart, lung, kidney, liver, intestine, pancreas, any tissue as muscle and skin, bone, blood vessel, lens, and stem cells of all kinds and origin, can be, for example, perfused with or soaked/bathed/immersed in (e.g., during transport) ischemia-reperfusion injury protecting composition alone or as part of an organ preservation solution.

It should be appreciated by those of skill in the art that the ischemia-reperfusion injury protecting composition can be provided in a syringe barrel already resuspended, provided in a dry powder form for resuspension prior to use, or provided in dry powder form with the syringe barrel also containing the solvent or solvents for resuspending the dry powder.

It is advantageous to formulate the ischemia-reperfusion injury protecting composition in dosage unit form for ease of administration and uniformity of dosage. Dosage unit form as used herein refers to physically discrete units suited as unitary dosages to be administered to an individual, with each unit containing a predetermined quantity of the ischemia-reperfusion injury protecting composition to produce the desired therapeutic effect.

Having now generally described the invention, the same will be more readily understood through reference to the following examples which are provided by way of illustration and are not intended to be limiting of the present invention.

### EXAMPLE 1

### Synthesis of Compounds #1, #2, #4 and #6

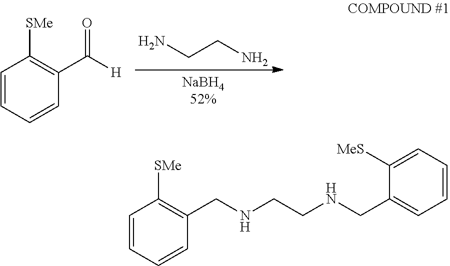

To a solution of 2-thiomethyl aldehyde (1.00 g, 6.57 mmol, 2.0 equiv), and ethylenediamine (0.22 mL, 3.30 mmol, 1 equiv) in ethanol (15.0 mL) was added titanium isopropoxide (1.96 mL, 6.34 mmol, 2.0 equiv). The solution was cooled to 0° C., and sodium borohydride (292 mg, 7.71 mmol, 2.3 equiv) added. After 2 h, the excess sodium borohydride was quenched with 0.5 M HCl (40 mL), and the solution partitioned with ethyl acetate. The organic layer was collected and the aqueous layer taken to pH>9 with 1M NaOH. The aqueous phase was then extracted with ethyl acetate (3×20 mL). The combined organic layers were dried (Na-2SO4), filtered, and concentrated under reduced pressure. The residue was purified via column chromatography (eluent: 30% CMA in dichloromethane, CMA=80:18:2 mixture of chloroform:methanol:ammonium hydroxide) to obtain the diamine (566 mg, 52%) as a yellow oil.

NMR: (D₂O, 400 MHz) δ 7.34 (d, 1H, J=7.2), 7.11-7.26 (m, 3H), 3.88 (s, 2H), 2.80 (s, 2H), 2.50 (s, 3H)

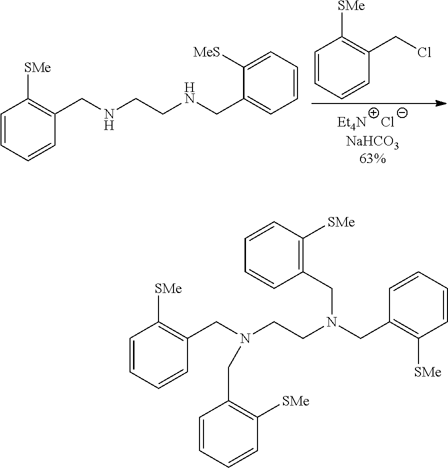

To a solution of diamine (515 mg, 1.5 mmol, 1 equiv), sodium bicarbonate (0.419 mg, 5.0 mmol, 3.3 equiv), tetrabutylammonium bromide (0.080 mg, 0.49 mmol, 0.3 equiv) in a biphasic mixture of dichloromethane (4.0 mL) and water (3.0 mL) was added benzyl chloride (0.58 mg, 3.4 mmol, 2.2 equiv). After 24 h, water (10 mL) and dichloromethane were added, and the organic layer extracted. The aqueous layer was further extracted with dichloromethane (1×10 mL). The combined organic layers were dried over magnesium sulfate, were filtered, and were concentrated. The residue was purified via flash column chromatography (eluent: 100% dichloromethane→10% ethyl acetate/dichloromethane) to provide the tetrabenzyl diamine (637 mg, 63%) as a white solid.

NMR: (D₂O, 400 MHz) δ 7.48 (d, 1H, J=8.0 Hz), 7.17-7.18 (m, 2H), 7.05 (m, 1H), 3.66 (s, 2H), 2.74 (s, 1H), 2.41 (s, 3H).

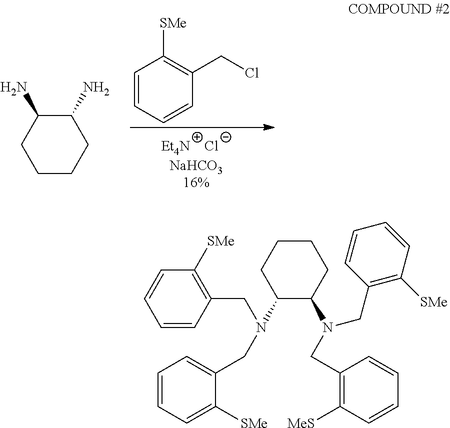

To a solution of diamine (96 mg, 0.84 mmol), sodium bicarbonate (360 mg, 4.3 mmol, 5 equiv), tetraethyl ammonium bromide (44 mg, 0.27 mmol, 0.25 equiv), in dichloromethane (2.0 mL) and water (2.0 mL) was added the alkyl chloride (0.73 mg, 4.2 mmol, 5 equiv). After 3 d, water (10 mL) and dichloromethane (10 mL) were added, and the organic layer removed. The aqueous layer was extracted further with dichloromethane (2×10 mL) and the residue purified via column chromatography (20% solution of CMA in dichloromethane, where CMA is an 80:18:2 solution of chloroform:methanol:ammonium chloride) to provide tetrabenzylamine (90 mg, 16%) as a yellow oil.

1H NMR (D₂O, 400 MHz) δ 7.83 (d, 4H, J=6.8 Hz), 7.15-7.28 (m, 8H), 6.80 (br, 4H), 3.80 (d, 4H, J=15.2 Hz), 3.52 (d, 4H, J=15.2 Hz), 2.67 (d, 2H, J=8.8 Hz), 2.42 (s, 12H), 2.37 (d, 2H), 1.76 (d, 2H), 1.2-1.6 (m, 4H).

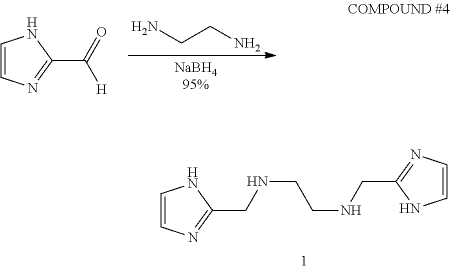

To a 250 mL round bottom flask containing a suspension of 2-formyl imidazole (2.99 g, 31.1 mmol 3 equiv) in methanol (60 mL) was added ethylenediamine (0.64 mL, 10.3 mmol, 1 equiv). The resulting faint yellow suspension was heated to 50° C. After 1 h the solution was cooled to 0° C., and sodium borohydride (1.55 g, 41.1 mmol, 4 equiv) added. After 1 h, the yellow suspension was heated to 60° C. After 1 h, the clear yellow solution was removed from heat and the solvent removed by rotary evaporation under reduced pressure. The residue was absorbed onto silica gel (12.0 g) through evaporation of a methanol suspension under reduced pressure. Purification by flash column chromatography on silica gel (eluent: 40% methanol/5% ammonium hydroxide/55% dichloromethane) provided the diamine 1 (2.18 g 95%) as a yellow oil.

NMR: (D₂O, 400 MHz) δ 6.96 (s, 4H), 3.72 (s, 4H), 2.54 (s, 4H)

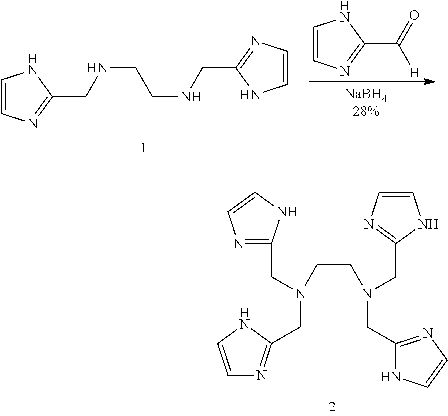

To a solution of diamine 1 (309 mg, 1.40 mmol, 1 equiv) and imidazole 2-carboxaldeyde (265 mg, 2.76 mmol, 2 equiv) in a solution of dichloromethane and acetic acid (12 mL, dichloromethane:acetic acid=5:1) at 0° C. was added sodium borohydride (99 mg, 2.67 mmol, 2 equiv). The suspension was stirred for 3 days, whereupon the reaction mixture was concentrated under reduced pressure. The residue was redissolved in methanol and absorbed onto silica gel (4.0 g) through concentration under reduced pressure. The absorbed crude reaction mixture was purified through flash column chromatography on silica gel (eluent: 30% methanol/5% ammonium hydroxide/65% dichloromethane) to provide tetra-imidazole 2 (149 mg, 28%) as a solid. The solid could be further purified through recrystallization from boiling water to provide fine needles. NMR (D₂O) δ 7.06 (s, 8H), 3.72 (s, 4H).

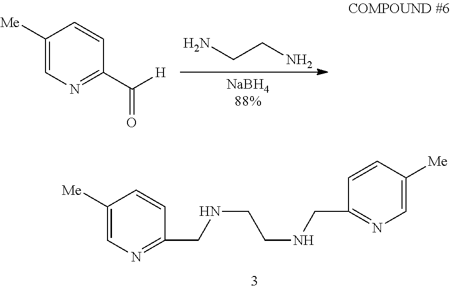

To a solution of 5-methyl-2-formyl pyridine (3.00 g, 24.4 mmol, 3.05 equiv) in methanol (50 mL) was added ethylenediamine (0.50 mL, 8.0 mmol, 1 equiv) at 23° C. The solution was heated to reflux. After 1 h, the reaction mixture was cooled to 0° C. and sodium borohydride (1.02 g, 26.9 mmol) was added. After a further 30 min at 0° C., the reaction mixture was heated to reflux. After 1 h, the reaction mixture was cooled to room temperature. Silica gel (12 g) was added, and the reaction mixture adsorbed onto silica gel through removal of the volatiles under reduced pressure. The absorbed mixture was purified through flash column chromatography on silica gel (eluent: 50% methanol/5% ammonium hydroxide/45% ethyl acetate) to provide the diamine 3 (1.94 g, 88%) as a clear yellow oil.

NMR (D₂O) δ 8.12 (s, 2H), 7.51 (d, 4H), 7.12 (d, 4H), 3.62 (s, 4H), 2.54 (s, 4H), 2.16 (s, 6H).

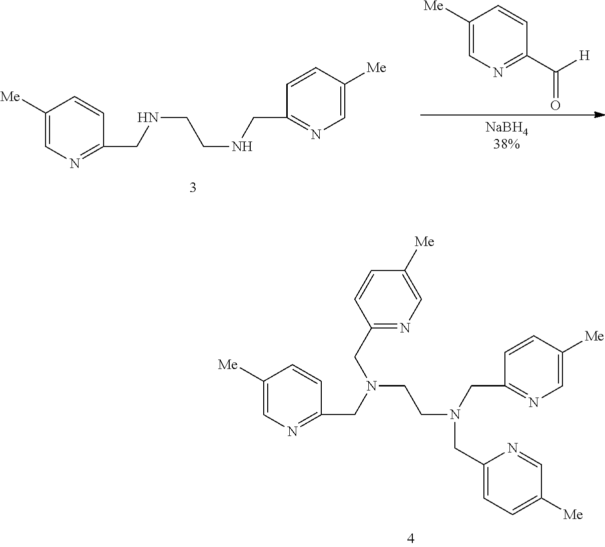 [SMILES: Cc1ccc(CNCCNCc2ccc(C)cn2)nc1 ; Cc1ccc(C=O)nc1 ; Cc1ccc(CN(CCN(Cc2ccc(C)cn2)Cc2ccc(C)cn2)Cc2ccc(C)cn2)nc1]

To a solution of diamine 3 (1.65 g, 4.34 mmol, 1 equiv) in a solution of dichloromethane and acetic acid (12 mL, dichloromethane:acetic acid=5:1) at 23° C. was added 5-methyl-2-formyl pyridine (1.52 g, 12.5 mmol, 3 equiv). The solution was cooled to 0° C. and sodium borohydride (678 mg, 17.9 mmol, 4 equiv) added. After 30 min the solution was heated to reflux. After 1 h the solution was cooled to room temperature and the crude reaction mixture filtered through a pad of silica gel (eluent: 5% ammonium hydroxide/35% methanol/60% dichloromethane). The solution was then concentrated under reduced pressure, and the residue purified by flash column chromatography on silica gel (eluent: 20% methanol/5% ammonium hydroxide/75% dichloromethane) to provide tetra-pyridine 4 (197 mg, 38%) as colorless crystals.

NMR (D₂O) δ 8.10 (s, 4H), 7.56 (d, 4H), 7.24 (d, 4H), 3.76 (s, 8H), 2.94 (s, 4H), 2.23 (s, 12H).

### EXAMPLE 2

### In Vitro Evaluation of the Potential Protective Effect of Compounds 4 and 6 Against Ischemic Insult in Neonate Rat Cardiomyocytes Cells Compared to N,N,N′,N′-tetrakis-(2-pyridylmethyl)-ethylenediamine (TPEN)

### In Vitro Protocol

### Preparation of Rat Heart Cell Cultures

Rat hearts (1-2 days old) were sterilely removed and bathed three times in Ca²⁺- and Mg²⁺-free PBS to remove excess blood cells. The hearts were minced to small fragments and then agitated in a proteolytic enzyme-RDB solution prepared from a fig tree extract as described previously (Brik et al., 1990; Shneyvays et al., 2001). The RDB was diluted 1:200 in PBS at 25° C. for a few cycles of 10 min each. The supernatant suspension containing dissociated cells, to which medium containing 10% horse serum was added, was centrifuged at 500 g for 5 min. After centrifugation, the supernatant phase was discarded, and cells were resuspended in Dulbecco's modified Eagle medium supplemented with 10% heat-inactivated horse serum and 0.5% chick embryo extract. Cell suspensions were diluted to 1×10⁶ cells/ml, and 300 μl was placed in 24 well plates (4 plates) collagen/gelatin-coated plastic culture dishes. Cultures were incubated in a humidified atmosphere of 5% CO₂ and 95% air at 37° C. A confluent monolayer, which exhibits spontaneous contractions in each well, developed in culture within 2 days. The growth medium was replaced every 3 days. The experiments were performed in vitro on these cardiomyocyte cultures after 3 to 5 days.

### Hypoxia Induction

Approximately 3×10⁵ cardiomyocytes/well from neonatal rat hearts was cultured and exposed to ischemia (oxygen and glucose deprivation) for 150 min. Prior to the exposure to ischemia, the growth medium was replaced with glucose-free PBS (250 μl/well). Ischemia was performed in a closed chamber by replacement of air with 100% Argon.

Ischemic Stress Injury Characterized by Lactate Dehydrogenase (LDH) Released from Cells and by the Propidium Iodide (PI) Staining Method

After 150 min of ischemia, a sample of 25 μl supernatant was taken from each well for LDH measurement. Thereafter, glucose (at final concentration of 4.5 g/L, by adding 5.6 μl from a stock solution of 200 mg/ml per well) was added to the plates containing ischemia-exposed cells, and the plates were transferred to a humidified incubator with 5% CO₂, 95% air at 37° C. for an additional 180 min, for reoxygenation. PBS supernatant samples (25 μl/well) were collected from each well after 1, 2 and 3 h for LDH measurement.

### LDH Activity Measurement

LDH activity was determined using LDH-L kit (Thermo Trace, Melbourne, Australia) within the cardiac cell medium (PBS). Medium (PBS) samples (25 μl) were collected at the end of the ischemic insult (after 150 min) and after 1, 2 and 3 h of reoxygenation. Samples from the plates exposed in parallel to normoxia were collected for the same tests. LDH enzymatic activity was measured in a spectrophotometer at a wavelength of 340 nm at 30° C.

### PI Staining Measurement

Cultured cardiomyocytes were incubated with propidium iodide (5 μg/ml) (Molecular Probes, Eugene, Oreg.) in PBS for 30 min at 37° C., and propidium iodide (PI) staining was performed by incubating the cells with PI (5 μg/ml) (Molecular Probes, Eugene, Oreg.) in PBS for 30 min at 37° C. in order to differentiate between dead and survived cells following hypoxia (PI-fluorescent stain only nucleus of damaged cardiomyocytes). Thereafter, cells were bathed with PBS twice, and the cell fluorescence was measured through excitation at 485 nm and emission at 635 nm.

### Experimental Design

For each compound, two similar plates were cultured. The same treatments were applied for both plates: one plate was exposed to ischemia and the other one was exposed to normoxia for total LDH cell evaluation. The effect of various concentrations (30, 100 and 300 nm) of Compound #4 and Compound #6 were compared with 1 μM TPEN as a reference compound. After adding the various compounds, all plates were gently shaken.

### Results

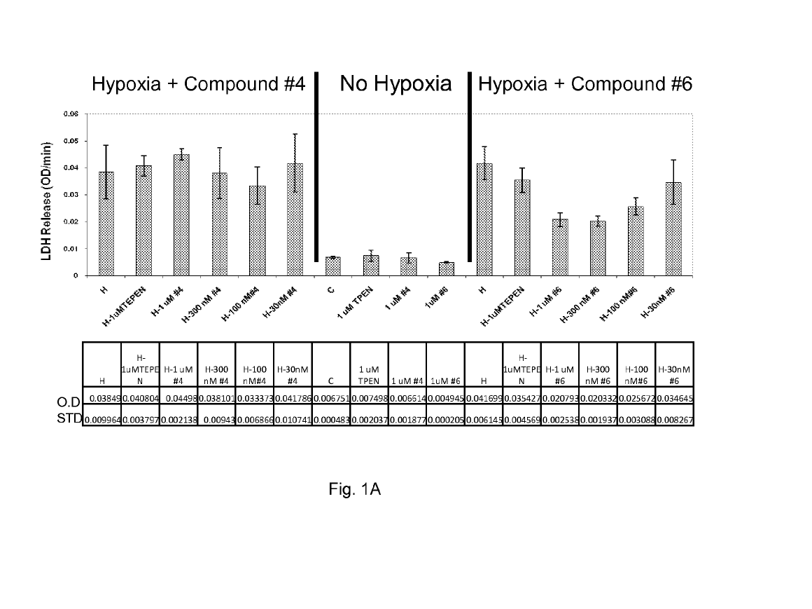

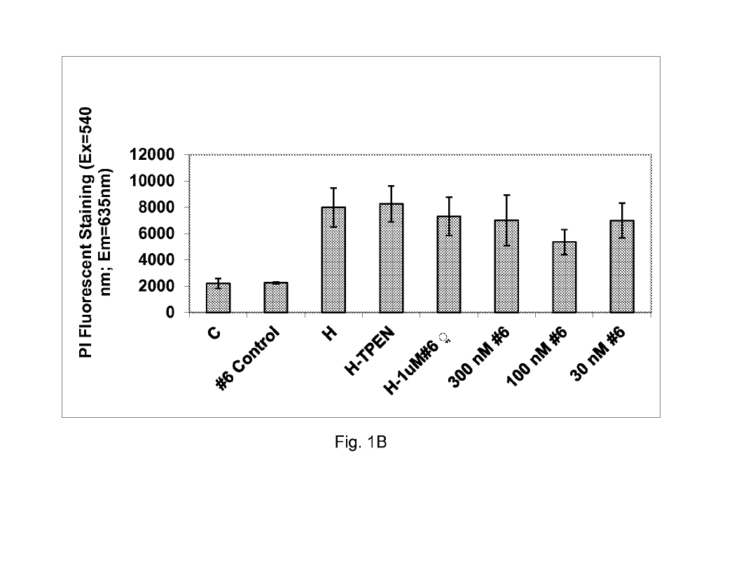

FIG. 1A shows the protective effects of compounds #4 and #6 against hypoxic insult in neonate rat cardiomyocyte cells as compared with the reference compound TPEN. While compound #4 was only slightly more effective than TPEN in attenuating cell injury, compound #6 clearly revealed significantly better protective outcome from hypoxia than TPEN, for the same test parameter.

FIG. 1B shows the protective effects of compound #6 from hypoxic insult in neonate rat cardiomyocyte cells evaluated by PI cells staining. After 3.5 hours of reoxygenation, all concentrations of compound #6: 1 μM, 300 nM, 100 nM and 30 nM revealed better protection than 1 μM of TPEN from cultures hypoxic injury as detected by PI nuclei staining, while 100 nM of #6 showed improve protection vs. other #6 concentrations.

### EXAMPLE 3

### In Vitro Evaluation of the Potential Protective Effect of Compounds 4 and 6 Against Ischemic Insult in PC12 Cells Compared to TPEN and TEMPOL

### In Vitro Protocol

### Preparation of PC12 Cells Cultures.

PC12 cells were grown in 25 cm² flasks in Dulbecco's modified Eagles Medium (DMEM), supplemented with 7% fetal bovine serum (FBS), 7% horse serum, 10,000 U/ml penicillin and 100 μg/ml streptomycin. Cells were grown in humidified incubator 5% CO₂, 95% air at 37° C. The medium was replaced every 2 days, and upon 70% confluence, the cells were counted and seeded for the ischemia experiments.

All cell growth, medium replacements and additions of compounds were carried out in a clean room according to ISO7 requirements (10,000 particles/m²).

### Hypoxia Induction

Approximately 1×10⁶ PC12 cells/well were cultured and exposed to ischemia (oxygen and glucose deprivation=OGD) for 4.5 hr. Prior to the exposure, the growth medium was replaced with glucose-free DMEM (1 ml/well). Ischemia was performed in a closed chamber by replacement of air with 95% nitrogen/5% CO₂.

Ischemic stress injury was characterized by lactate dehydrogenase (LDH) released from cells. After 4.5 hr of ischemia, a 50 μl sample of supernatant was taken from each well for LDH measurement. At the end of the ischemic insult (after 4.5 hr), glucose (at final concentration of 4.5 g/L by adding 10 μl from stock solution of 450 mg/ml per well (1 ml)) was added to the plates which underwent ischemia. The plates were then transferred to a humidified incubator and exposed to 95% air/5% CO₂ at 37° C. for additional 18 hr of reoxygenation. DMEM supernatant samples (25 μl/well) were collected from each well for LDH measurements. The samples were centrifuged at 3500 rpm for 5 min, and 5 μl aliquots of supernatant was taken for LDH measurement.

For every experiment, a duplicate plate was prepared as a control study under normoxia. Cells were kept in a humidified incubator (5% CO₂ and 95% air at 37° C.) in cell culture complete medium at a volume of 1 ml for 18 hr, and the same compounds were added with the same concentrations. Supernatant was collected for LDH measurement as indicated in the LDH activity measurement section below. Afterwards (at the end of the reoxygenation phase), the plates were frozen (at −70° C. for 30 min) and thawed once. 25 μl/well sample was collected and the samples were centrifuged at 3500 rpm for 5 min and then taken for LDH measurement (total).

### LDH Activity Measurement

LDH activity was determined using LDH-L kit (Pointe Scientific Inc., Michigan, USA) in the PC12 cell medium. Medium samples (5 μl) were collected at the end of the ischemic insult (after 4.5 hr) and after the reoxygenation period. In parallel, samples from the plates exposed to normoxia were collected. LDH enzymatic activity was measured in a spectrophotometer at a wavelength of 340 nm at 37° C. Data analysis was performed as follows: [OD (cycle 3)−OD (cycle 1)experiment]−[OD(3)−OD(1) control])/[OD(3)−OD(1) total]×100, and presented as percentage of total cell death.

To present the results in IU/L (international unit/liter), which is defined as the amount of enzyme that catalyzes the transformation of one micromole of substrate per minute, the change in absorbance was multiplied by 6592.

### Experimental Design

### Pilot Calibration Study on Protective Effect of the Positive Controls Tempol (4-hydroxy-2,2,6,6-tetramethylpiperidine-1-oxyl) and TPEN Against Ischemia Induced Cell Death

One day before the experiment, 12-well plates were coated with 200 μg/ml type I rat tail collagen (BD Biosciences, Bedford, Mass.) and were further sterilized under UV. 1×10⁶ PC12 undifferentiated cells/well were seeded on the plates and allowed to adhere overnight under regular conditions (DMEM medium high glucose 4.5 mg/ml, 5% CO₂ and 95% air at 37° C.). Each experiment included 4 plates, two for normoxia and two for ischemia which underwent the same following treatment for all 4 plates.

A half hour before the onset of the ischemic insult, the antioxidant Tempol (4-hydroxy-2,2,6,6-tetramethylpiperidine-1-oxyl) (Sigma, St. Louis, Mo.) at a concentration of 0.5 mM (10 μl/well taken from a 8.6 mg/ml stock solution) or TPEN at a concentration of 3 μM and 10 μM were added to the plates. Before the experiments, the medium was replaced with DMEM glucose free medium at 1 ml volume/well. TPEN or 10 μl Tempol were again added to the appropriate wells and was maintained throughout all experiments.

Thereafter, the plates were introduced into a device inducing ischemia atmosphere with 95% nitrogen and 5% CO₂ for 4.5 hr. Oxygen level was monitored online by a digital oxygen monitor showing up to 1%. At the end of OGD, 25 μl from each well was collected into 0.5 ml tubes for the LDH assay. 4.5 mg/ml glucose was immediately added to each well and the cells were transferred to an incubator with 5% CO₂ and 95% air at 37° C. for 18 hr. After the reoxygenation period, 25 μl was taken from each well for the LDH monitoring. The plates were then frozen (−70° C. for 30 min) and afterwards thawed once for LDH evaluations of total cell death.

### Compounds #4 and #6 Tested in the PC12 Ischemic Model

Two compounds (Compounds #4 and #6) were tested by the PC12 ischemic model. Each compound was tested at different concentrations: 300 nM, 1 μM, 3 μM. For each compound, four similar plates were cultured. Two plates were exposed to ischemia and the other two served for normoxia. The same treatments were applied for all plates and for total LDH cell evaluation, and plates were gently shaken following addition of compounds. Overall, 4 plates served for each compound, and all experiments were repeated twice.

### Results

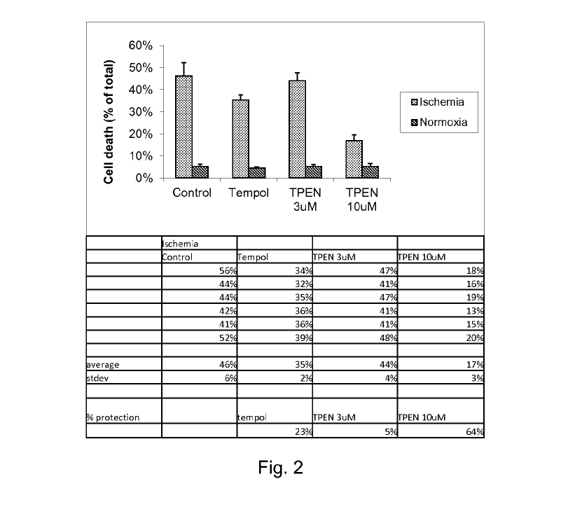

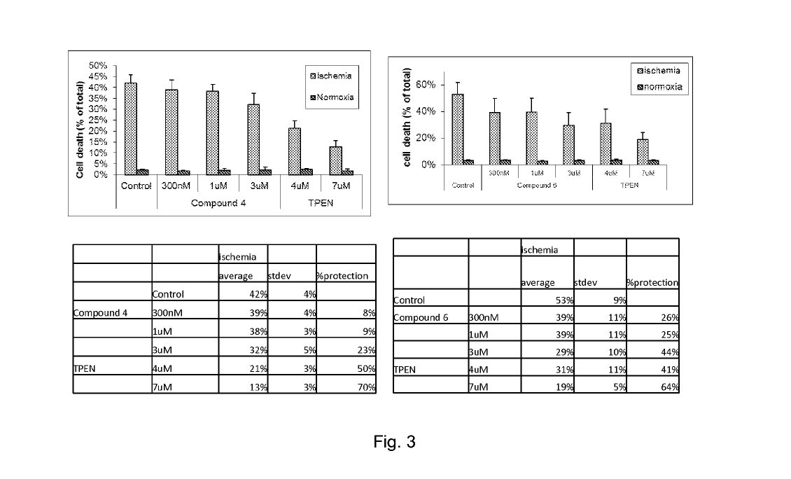

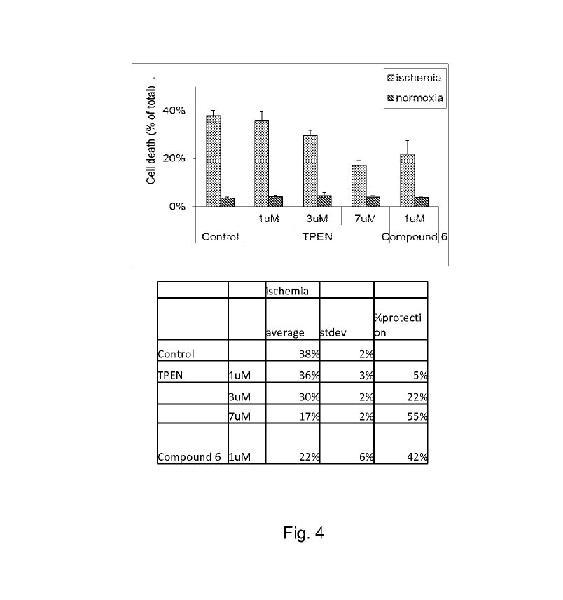

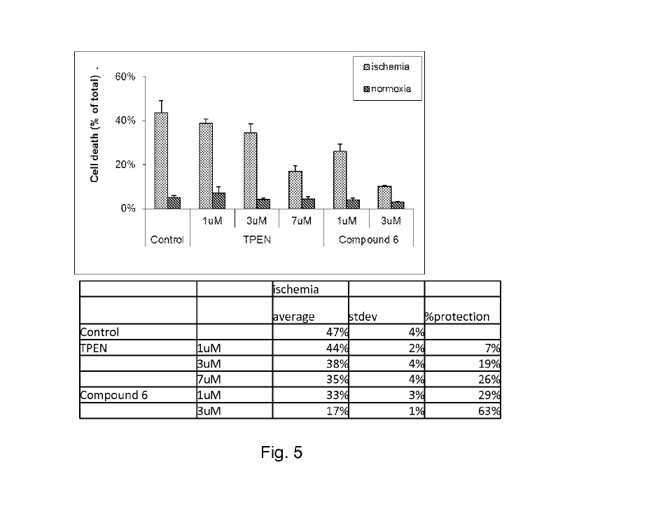

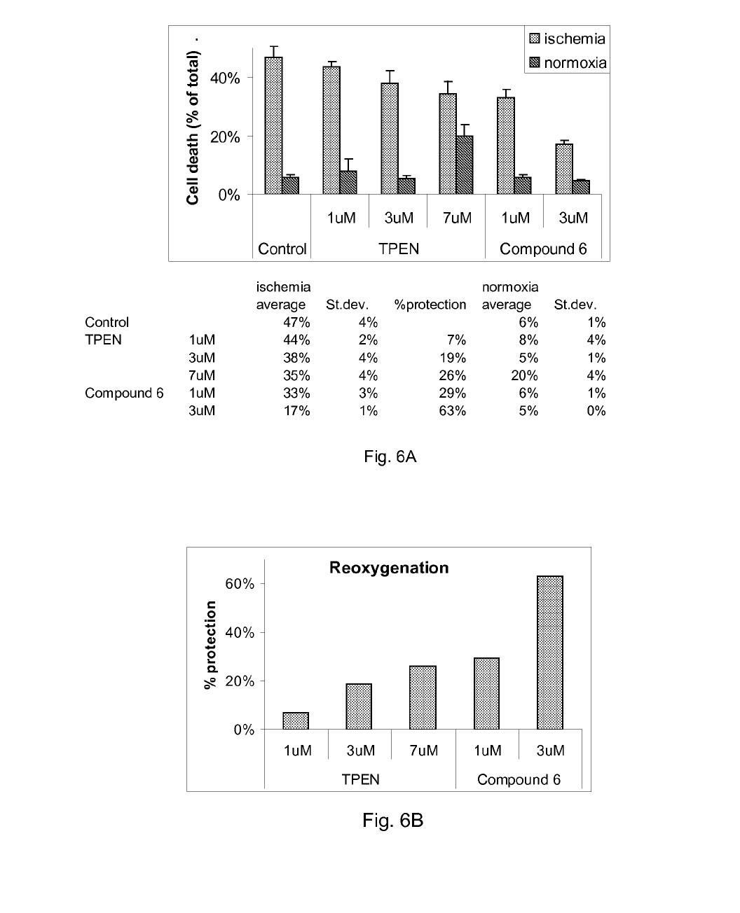

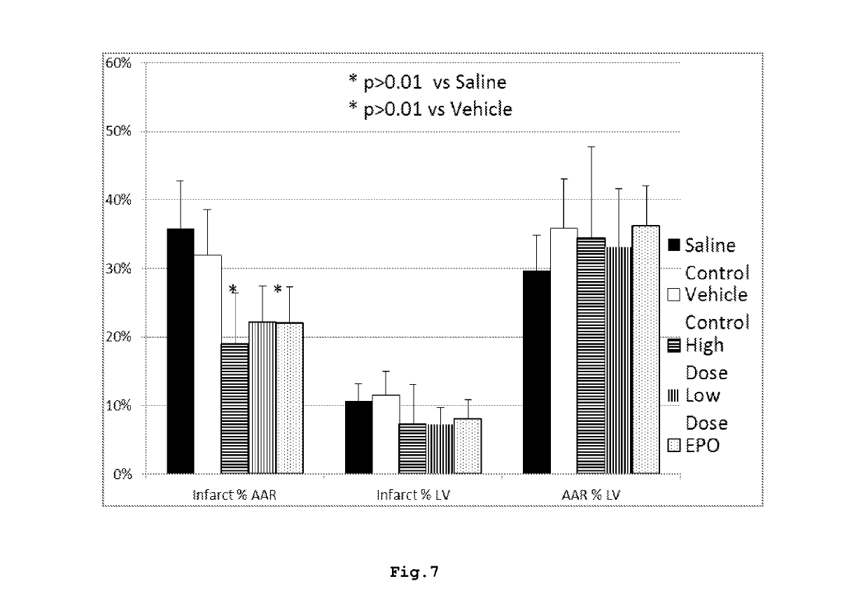

The protective effects of Compounds #4 and #6 vs. the reference compound TPEN and the antioxidant Tempol, from ischemic insult in PC12 cells are shown in FIGS. 2-5. FIG. 2 shows that TPEN is effective at a concentration of 10 μM. in reducing death of PC12 cells subjected to 4.5 hours ischemia. It is also significantly effective vs. Tempol, a strong antioxidant. FIG. 3 shows the comparative effect of Compound #6 vs. Compound #4 relatively to the effects of TPEN as a positive control: Compound #6 was found to be more effective than Compound #4 at similar concentrations. In addition, Compound #6 also exhibited similar protection with TPEN from cell death under lesser concentrations. FIG. 4 shows that 1 μM Compound #6 is significantly more potent then TPEN under the same concentration and also more effective than 3 μM TPEN in protecting PC12 cells against ischemia insult-induced cell death. Furthermore, FIG. 5 shows that while Compound #6 at a concentration of 1 μM is 2-fold more effective than 1 μM TPEN, at 3 μM it is 3-fold more effective than TPEN at the same concentrations and 4-fold relative to the un-protected control group of cells. FIG. 6A depicts dose response function of selected concentrations of compound #6 and TPEN, clearly showing that the former is superior to the latter, in attenuating cell death following ischemia-reperfusion. FIG. 6B illustrates the protection percentile of these 2 compounds of the same study demonstrating 29% and 63% protection from cell death vs. 7% and 19% only obtained by TPEN under identical concentrations, respectively.

### EXAMPLE 4

### In Vivo Effects of Compound #6 on Cardiac Function, Infarct Size, and Troponin I Levels in a Rat Animal Model of Ischemia/Reperfusion

This study was conducted to evaluate the effects of the Compound #6 on cardiac function, infarct size, and Troponin I levels in a model of ischemia/reperfusion in the CD® (CR1CD® (SD)) rat. Phase A was conducted for toxicity evaluation purposes. In Phase B (the efficacy phase), two treatment groups of eight male rats were administered Compound #6 at dose levels of 0.2 and 2 mg/kg via intravenous infusion following myocardial infarction. The 2 mg/kg dose of Compound #6 was administered intravenously via a slow infusion starting 3 to 14 minutes prior to reperfusion and continuing for 20 minutes to 20 minutes and 32 seconds, which was approximately 6 to 17 minutes after reperfusion (depending on the infusion start time). The 0.2 mg/kg dose of Compound #6 was administered intravenously via a slow infusion starting 10 minutes prior to reperfusion and continuing for 30 minutes to 30 minutes and 30 seconds, which was approximately 20 minutes after reperfusion.

The animals in Phase B were monitored for selected cardiovascular endpoints from at least 15 minutes prior to acute myocardial infarction until at approximately 1 hour post reperfusion. Blood samples for the evaluation of Troponin I were collected from all surviving Phase B animals prior to coronary artery occlusion and at termination. At study termination, the Phase B animals were submitted to necropsy for limited necropsy examinations and infarct analysis.

### Materials and Methods

### Vehicle, Positive Control Article, and Compound #6 Preparation.

Fresh vehicle, 0.9% Sodium Chloride for Injection, USP (saline), was dispensed for use on the day of each administration and was stored refrigerated at 2 to 8° C.

Fresh vehicle for Compound #6, 10% (w/v) 2-hydroxypropyl-beta-cyclodextrin and 100 Mm sodium chloride in deionized water, was prepared once for use on study by mixing the appropriate amounts of each vehicle component and adjusting the pH to 5.06 to 5.66 using 2N HCl. The prepared vehicle was filtered through a 0.22 μM PVDF syringe filter, aliquoted and was stored refrigerated at 2 to 8° C. in amber glass serum bottles. The prepared vehicle was dispensed for use on the day of each administration and was refrigerated at 2 to 8° C.

Fresh positive control article, erythropoietin, was dispensed for use on the day of each administration and was stored refrigerated at 2 to 8° C.

Formulations of Compound #6 were prepared once on the day prior to administration in Phase A and on the day of each administration in Phase B at nominal concentrations of 0.2 and 2.0 mg/Ml in 10% (w/v) 2-hydroxypropyl-beta-cyclodextrin and 100 Mm sodium chloride in deionized water. Separate preparations were made for each nominal concentration, and the pH was adjusted to 5.14 to 5.98 using 2N HCl. The prepared test article was mixed by magnetic stirring for at least 60 minutes to achieve a clear solution, was filtered through a 0.22 μM PVDF syringe filter, and stored refrigerated at 2 to 8° C. in amber glass serum bottles.

### Induction of Myocardial Infarction Procedure.

Phase A of the study was conducted using routine aseptic technique. It was not necessary for aseptic technique to be used in Phase B as the surgery was a non-survival surgery, and animals were not recovered from anesthesia.

The animals were placed on mechanical ventilation and not intubated. Once anesthetized, the animal was placed in a right lateral recumbency, an arterial catheter or polyethylene tube was placed in the carotid artery for monitoring blood pressure and a thoracotomy was performed. The left coronary artery (LCA) was identified. A ligature was placed around the LCA and the vessel was ligated. The LCA was maintained occluded for 29 to 43 minutes. Occlusion of the vessel was verified via blanching of the heart and via ST interval changes. At the end of the occlusion period, the knot was loosened leaving the ligature in place, and the vessel was allowed to reperfuse for 3 hours 27 minutes to 4 hours 51 minutes. Following infarct, a chest tube was placed, the deep and superficial muscle layers were sutured at the surgical incision, and the skin was closed with staples. The chest tube was removed after spontaneous respiration was established. In Phase A, the animals were recovered from anesthesia and evaluated for 4 days. In Phase B, animals were maintained under anesthesia for 3 hours 27 minutes to 4 hours 51 minutes prior to being submitted to necropsy.

### In Vivo Administration.

In Phase A, Compound #6 was administered once on Day 0 via intravenous infusion at a dose level of 2 mg/kg. The dose was administered at a dose volume of 1 mL/kg (0.05 mL/kg/minute). Compound #6 was administered intravenously via a slow infusion starting 10 minutes before and continuing until 10 minutes after reperfusion.

In Phase B, Compound #6 was administered once on Day 0 via intravenous infusion at dose levels of 0.2 and 2 mg/kg. The doses were administered at a dose volume of 1 mL/kg (0.05 [2 mg/kg] or 0.033 [0.2 mg/kg] mL/kg/minute). Two control groups received the vehicle (saline) or the vehicle for Compound #6 (10% (w/v) 2-hydroxypropyl-beta-cyclodextrin and 100 mM sodium chloride in deionized water) in the same manner as the 2 mg/kg treated group. The vehicles and 2 mg/kg Compound #6 were administered intravenously via a slow infusion starting 3 to 14 minutes prior to reperfusion and continuing for 20 minutes to 20 minutes and 32 seconds, which was approximately 6 to 17 minutes after reperfusion (depending on the infusion start time). The 0.2 mg/kg Compound #6 was administered intravenously via a slow infusion starting 10 minutes prior to reperfusion and continuing for 30 minutes to 30 minutes and 30 seconds, which was approximately 20 minutes after reperfusion. The positive control article, Erythropoietin, was administered at 5000 IU/kg via a bolus intravenous injection. The positive control article dose was administered at a dose volume of 2.5 mL/kg. The positive control was administered via a 3 to 60 second injection at 5 minutes prior to reperfusion.

### In-Life Examinations

### Cageside Observations

All animals were observed for morbidity, mortality, injury, and the availability of food and water twice daily.

### Detailed Clinical Observations

In Phase A, a detailed clinical examination of each animal was performed at 1, 2, and 4 hours postdose and daily thereafter for 3 days. In Phase B, detailed clinical examinations were not performed. The observations included, but were not limited to, evaluation of the skin, fur, eyes, ears, nose, oral cavity, thorax, abdomen, external genitalia, limbs and feet, respiratory and circulatory effects, fecal output, autonomic effects such as salivation, and nervous system effects including tremors, convulsions, reactivity to handling, and unusual behavior.

### Body Weights

In Phase A, body weights for all animals were measured and recorded at receipt, prior to randomization, just prior to surgery, and daily for 3 days during the study. In Phase B, body weights for all animals were measured and recorded at receipt, prior to each randomization as applicable, and just prior to surgery, except that a body weight was not recorded prior to randomization for the animals received in the second shipment. In both phases, the body weights recorded at receipt and prior to randomization are not reported but are maintained in the study file.

### Cardiovascular Monitoring

In Phase B, animals were anesthetized for monitoring of selected cardiovascular parameters. For experimentation, the blood pressure catheter implanted in the carotid artery was attached to an external transducer and data acquisition was started. Blood pressure (systolic, diastolic, and mean arterial) and heart rate (derived from blood pressure) were monitored from at least 15 minutes prior to acute myocardial infarction (AMI) until at least 1 hour after reperfusion. Individual animal cardiovascular data tables are not reported but are maintained in the study file.

### Cardiac Troponin Concentration Analysis

In Phase B, cardiac troponin I evaluations were conducted at 3 to 54 minutes prior to coronary artery occlusion and prior to termination on all animals. The animals had access to drinking water and food prior to sample collection. Blood samples (approximately 1.2 mL) were collected from the carotid catheter or tail vein (prior to occlusion) or the vena cava (termination). The samples were collected into tubes containing serum separators with no anticoagulant. The samples were stored frozen at −50 to −90° C. until analyzed.

### Postmortem Study Evaluations

At study termination for Phase A, the animals were euthanized via carbon dioxide inhalation. Euthanasia was confirmed via cervical dislocation and the carcasses were discarded.

Phase B animals were submitted for infarct analysis. Limited necropsy examinations were performed under procedures approved by a veterinary pathologist on all surviving animals at the scheduled necropsy.

### Infarct Analysis

After LAD reperfusion (4 hours), the animals were euthanized by an intraperitoneal injection of sodium pentobarbital followed by exsanguination via severing the abdominal vena cava. The heart was flushed with heparinized Lactated Ringers Solution (LRS) until clear of blood and the LAD was tied off using the same ligature used to occlude the vessel and Evan Blue dye was injected. The heart was weighed and breadloafed (4-5 sections per animal). Next the thickness of each slice was recorded and photographed for measurement and evaluation of the Area at Risk or the area of the heart perfused by the occluded artery. The sections of the heart were weighed and stained with a solution of 1% triphenyltetrazolium chloride (TTC) in pH 7.4 sodium phosphate buffer for 20 minutes at 37° C., and photographed a second time for measurement and evaluation of the infarct. Following staining, the normal heart tissue was blue/purple, the area at risk was red, and the infarct was white. The infarct size was calculated as a percent of the total Area At Risk (AAR). A ruler with millimeter units or a calibrated measurement block were imaged in the same condition to calibrate the infarct area. The images including the ruler were analyzed with ImageJ 1.32j software from NIH. The ratios of infarct/AAR, infarct/LV and AAR/LV were calculated. Once the final photographs were obtained, the heart sections were placed in 10% neutral buffered formalin (NBF) and saved for possible future analysis.

### Results and Discussion

### Clinical Outcomes

### Mortality

Phase A: All animals survived to the scheduled necropsy.

Phase B: A total of 53 rats were submitted to surgery for induction of AMI. Of the 53 rats that underwent AMI, 13 animals (25%) either died during the surgical procedure or during the first 2 hours following reperfusion. In addition, six Group (saline) animals, one Group 3 (vehicle) animal, and two Group 5 (0.2 mg/kg) animals died between 3 and 4 hours post reperfusion. These animals were included in the study as adequate time had passed for the infarct to develop. The differences in mortality rates between the saline animals and the treated animals suggest a cardioprotective effect of Compound #6.

### Animal Observations

Phase A: Clinical findings were limited to observations on the day of surgery and the day following surgery and were considered to be within normal limits for animals recovering form thoracic surgery. Body weights in these animals declined over the course of the study, but remained within normal ranges for animals of this age and strain.

Phase B: All animals placed on study were considered to be in good health and were in approximately the same weight range.

### Cardiovascular Evaluations

### Systolic, Diastolic, and Mean Arterial Blood Pressures

Blood pressures were monitored continuously in all animals beginning 15 minutes prior to AMI through 1 hour post-reperfusion. During the pre-occlusion period, mean blood pressures were slightly higher in animals in the Compound #6 and positive control groups. Following Occlusion (Time 0 on graphs), blood pressure in the Compound #6 and positive control animals decreased to levels similar to control animals. Within 5-10 minutes of reperfusion, mean arterial pressures increased in animals treated with both 0.2 mg/kg of Compound #6 and the positive control article. This increase corresponds to the bolus injection in the positive control article animals. In animals treated with 2 mg/kg of Compound #6, blood pressure reached peak levels approximately 20 minutes post reperfusion or approximately 5 minutes following the completion of the infusion. Mean pressures in the saline control animals remained unchanged post reperfusion and for the vehicle control animals mean pressures returned to baseline levels. By the end of the monitoring period, there were no differences between treatment groups.

### Heart Rate

Heart rate was monitored continuously in all animals beginning 15 minutes prior to test or control article administration through 1 hour post-reperfusion. During the pre-AMI, occlusion and post reperfusion periods, mean heart rates were similar across dose groups.

### Cardiac Troponin

Troponin I (TnI) was evaluated prior to occlusion and again prior to termination. The results of this analysis found troponin levels to be variable within groups. Animals treated with Compound #6 at dose 0.2 and 2.0 mg/kg were noted to have substantially lower TnI levels than both saline and vehicle control animals. These results, while not statistically different, support the infract size analysis and suggest less damage occurred in treated versus control animals. Animals treated with the positive control were found to have TnI levels similar to vehicle control animals.

### Infarct Evaluation

The infarct data are presented below in Table 1. At the end of the 4 hour reperfusion interval, the animals were submitted to necropsy, and the ligature was re-tied around the coronary artery. The heart was then removed and dye was injected through the aorta. The heart was observed for adequate staining of the normal myocardium and then cut into slices, photographed, and placed in TTC for staining of the infarct. The heart was again photographed and then placed in formalin. Morphometric analysis of the photos of the hearts found the area at risk as a percentage of the left ventricle (LV) to be similar between all dose groups and controls. Analysis of the infarct size as a percent of the area at risk found that administration of 2 mg/kg Compound #6 as an infusion produced a significant decrease in infarct size when compared to saline control and vehicle controls (19.1±7.38% vs 35.9±6.84% p=0.0004 and 31.9±6.70% p=0.003 respectively). In addition, the 0.2 mg/kg of Compound #6 as an infusion produced a significant decrease in infarct size when compared to saline control and vehicle controls (22.2±5.24% vs 35.9±6.84% p=0.001 and 31.9±6.70% p=0.007 respectively). The positive control treated animals also demonstrated significantly less cardiac damage as compared to saline control animals.

TABLE 1
Summary of Infarct Data

| | | 0 mg/kg | 0 mg/kg | 2 mg/kg | 0.2 mg/kg | Positive |
|---|---|---|---|---|---|---|
| End- | | (Vehicle- | (Ve- | (Comp | (Comp | Control |
| point | | Saline) | hicle) | #6) | #6) | (EPO) |
| Infarct/ | Mean | 36 | 32 | 19^{b} | 22^{b} | 22^{b} |
| AAR (%) | SD | 6.8 | 6.7 | 7.4 | 5.2 | 5.2 |
| | N | 8 | 8 | 8 | 7* | 8 |
| AAR/ | Mean | 29 | 36 | 34 | 33 | 36 |
| LV (%) | SD | 5.1 | 7.3 | 13.2 | 8.4 | 5.8 |
| | N | 8 | 8 | 8 | 7 | 8 |
| Infarct/ | Mean | 11 | 12 | 7 | 7 | 8 |
| LV (%) | SD | 2.5 | 3.5 | 5.7 | 2.3 | 2.7 |
| | N | 8 | 8 | 8 | 7 | 8 |

N - Number of measures used to calculate the mean
SD - Standard Deviation
^{b}Significantly different from 0 mg/kg (Vehicle-Saline); (p<0.01)
*Animal 2460, heart overstained and could not be evaluated.

### Conclusion

The results of this study demonstrated that administration of either 0.2 or 2.0 mg/kg IV of Compound #6 as an infusion produced a significant decrease in infarct size following a 30 minute ischemia-reperfusion injury. The results are supported by substantially lower, but not significantly lower, Troponin I levels in these animals. Blood pressures were slightly elevated during infusion of Compound #6 at both dose levels, but returned to levels comparable to vehicle animals following completion of infusion. Heart rate was unaffected by Compound #6 administration. This study demonstrated that administration of either 0.2 or 2.0 mg/kg of Compound #6 produced a significant reduction in infarct size in a model of ischemia reperfusion in the SD rat. In addition, intravenous administration of Compound #6 at dose levels up to 2.0 mg/kg following myocardial ischemia was not associated with any mortality, clinical signs or changes in body weights during Phase A and was not associated with any adverse changes in blood pressure or increase in mortality in Phase B. The results of this study demonstrate that administration of Compound #6 at dose levels up to 2 mg/kg following myocardial ischemia is not associated with any toxicity in the rat.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the inventions following, in general, the principles of the invention and including such departures from the present disclosure as come within known or customary practice within the art to which the invention pertains and as may be applied to the essential features hereinbefore set forth as follows in the scope of the appended claims.

All references cited herein, including journal articles or abstracts, published or corresponding U.S. or foreign patent applications, issued U.S. or foreign patents, or any other references, are entirely incorporated by reference herein, including all data, tables, figures, and text presented in the cited references. Additionally, the entire contents of the references cited within the references cited herein are also entirely incorporated by reference.

Reference to known method steps, conventional methods steps, known methods or conventional methods is not in any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

### REFERENCES

Appelbaum Y, Csonka C, Blasig I E, Tosaki A., Role of nitric oxide and TPEN, a potent metal chelator, in ischemic and reperfused rat isolated hearts. Ferdinandy P, Clin. Exp Pharmacology. Physiol. 25(7-8):496-502 (1998 July-August).
Appelbaum Y. J., Bublil M., Borman J. B., Uretzky G. and Chevion M., “Role of the Metal Chelator TPEN against Ischemic and Reperfusion Injury in the Isolated Perfused Rat Heart”, Proceedings of the SOD V Conference, Jerusalem, Israel, p. 135, September. 17-22 (1989)
Appelbaum Y. J., Kuvin J., Borman J. B., Uretzky G., and M. Chevion, Role of Oxygen-Free Radicals in Reperfusion-Induced Arrhythmias: Protection by Neocuproine”, Free Radicals in Biology and Medicine, 8:133-143 (1990).
Appelbaum Y. J., Kuvin J., Chevion M. and Uretzky G., “TPEN, A Heavy Metal Chelator, Protects the Isolated Perfused Rat Heart from Reperfusion-Induced Arrhythmias”, Journal of Molecular and Cellular Cardiology, Vol. 20 (Supp. V), Abstract No. 32 (1988)
Appelbaum Y. J., Uretzky G., and Chevion M., “The Protective Effect of Neocuproine on Cardiac Injury Induced by Oxygen Active Species in the Presence of Copper Sulfate”, Journal of Molecular and Cellular Cardiology, Vol. 19 (Supp. III), Abstract No. 8, (1987)
Appelbaum Y. J., Chevion M., Borman J. B., Bublil M., Uretzky G J. Trace, Metal chelating agents attenuate post ischemic myocardial injury: A study of the isolated perfused rat heart. Elem. Electrolytes Health Dis. 4(3):195-196 (1990).
Avi L. Friedlich*, Mark A. Smith, Xiongwei Zhu, Atsushi Takeda, Akihiko Nunomura, Paula I. Moreira and George Perry, Oxidative Stress in Parkinson's Disease, The Open Pathology Journal 3:38-42 (2009)
Bressler, Joseph P Metal transporters in intestine and brain: their involvement in metal-associated neurotoxicities. Human & Experimental Toxicology 26(3):221-229 (2007)
Brik H, and Shainberg A. Thyroxine induces transition of red towards white muscle in cultured heart cells. Basic Res Cardiol 85:237-246 (1990)
Gang Liu, Ping Men, Peggy L. R. Harris, Raj K. Rolston, George Perry and Mark A. Smith, Nanoparticle iron chelators: A new therapeutic approach in Alzheimer disease and other neurological disorders associated with trace metal imbalance. Neuroscience Letters 406(3):189-1939 (October 2006)
Hochhauser E, Ben-Ari Z, Pappo O, Chepurko Y, Vidne B A., TPEN attenuates hepatic apoptotic ischemia/reperfusion injury and remote early cardiac dysfunction, Apoptosis 10(1):53-62 (January 2005).
Huang X, Moir R D, Tanzi R E, Bush A I, Rogers J T., Redox-active metals, oxidative stress, and Alzheimer's disease pathology, Ann N Y Acad Sci. 1012:153-63 (2004 March).
Jomova K, Valko M., Advances in metal-induced oxidative stress and human disease, Toxicology, 2011 May 10; 283(2-3):65-87. Epub 2011 Mar. 23.
Jomova Klaudia, Vondrakova Dagmar, Lawson Michael, Valko Marian, Metals, oxidative stress and neurodegenerative disorders, Mol Cell Biochem, 345:91-104 (2010)
Karck M., Appelbaum Y., Schwalb H., Haverich A., Chevion M. and Uretzky G., “TPEN, A Transition Metal Chelator, Improves Myocardial Protection during Ischemia”, Journal of Heart and Lung Transplantation 11:979-985 (1992).
Math P. Cuajungco, Kyle Y. FAGÉT, Xudon G Huang, Ruldolph E. Tanzi and Ashley I. Bush, Metal Chelating as a Potential Therapy for Alzheimer's disease. Annals of the New York Academy of Sciences 920:292-304 (2000)
Perez, Lissette R. and Franz, Katherine J., Minding metals: Tailoring multifunctional chelating agents for neurodegenerative disease, Dalton Trans., 39:2177-2187 (2010)
Robert Crichton, Roberta Ward, Metal-based Neurodegeneration: From Molecular Mechanisms to Therapeutic Strategies. ISBN: 978-0-470-02255-9 Hardcover, 238 pages (December 2005)
Shneyvays V, Mamedova L, Zinman T, Jacobson K, and Shainberg A. Activation of A₃ adenosine receptor protects against doxorubicin-induced cardiotoxicity. J Mol Cell Cardiol 33:1249-1261 (2001)
Tabner, Brian J. and El-Agnaf, Omar M. A. and German, M. J. and Fullwood, Nigel J. and Allsop, David, Protein aggregation, metals and oxidative stress in neurodegenerative diseases. Protein aggregation, metals and oxidative stress in neurodegenerative diseases, Biochemical Society Transactions, 33(5):1082-1086. ISSN 0300-5127 (2005)
Xudong Huang Robert D. Moir., Redox-Active Metals, Oxidative Stress, and Alzheimer's Disease Pathology. Annals of the New York Academy of Sciences (12 Jan. 2006)
Yelena A. Shmist¹, Roman Kamburg², Gal Ophir², Alex Kozak², Vladimir Shneyvays¹, Yori J. Appelbaum², Asher Shainberg^{1*}, TPEN Improves Myocardial Protection against Ischemia by Modulation of Intracellular Ca²⁺ Homeostasis, The Journal of Pharmacology And Experimental Therapeutics, 10.1124 (Jan. 28, 2005)

1. Compound #6 having the chemical formula

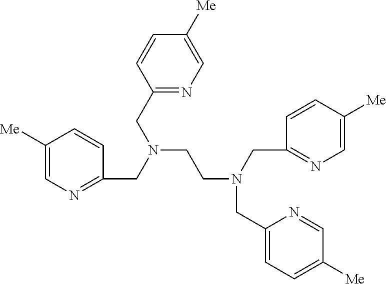 [SMILES: Cc1ccc(CN(CCN(Cc2ccc(C)cn2)Cc2ccc(C)cn2)Cc2ccc(C)cn2)nc1]

2. A composition, comprising the compound of claim 1 and a pharmaceutically acceptable excipient or carrier.

3. A tissue and organ preserving composition, comprising a solution which includes a tissue and organ preserving effective amount of the compound of claim 1 and a solvent.

4. A method for preventing or treating ischemia-reperfusion injury, comprising administering an effective amount of the compound of claim 1 to a patient at risk of ischemia-reperfusion injury.

5. The method of claim 4, wherein the patient is selected from the group consisting of an individual experiencing blood loss, an individual having a stroke or cardiopulmonary arrest, and an individual about to undergo or undergoing a surgical or medical procedure.

6. The method of claim 5, wherein the patient is an individual experiencing blood loss from trauma in an accident or in a battlefield.

7. The method of claim 5, wherein, in an individual about to undergo or is undergoing a surgical or medical procedure, a tissue or organ of the individual is bathed or immersed in a tissue and organ preserving composition comprising compound #6.

8. The method of claim 4, which protects the heart of a patient from ischemia-reperfusion injury.

9. The method of claim 4, wherein the patient is human.

10. A method for protecting and preserving an excorporeal tissue or organ from ischemia and oxygen radical-related damage, comprising immersing an excorporeal tissue or organ in an excorporeal tissue and organ protecting and preserving effective amount of the compound of claim 1.

11. The method of claim 10, wherein the excorporeal organ to be protected and preserved from ischemia and oxygen radical-related damage is the heart.

12. The method of claim 11, wherein the heart is a human heart.

13. A method for protecting cells from ischemia and oxygen radical-related damage, comprising immersing cells in a solution which includes an ischemia and oxygen radical-related damage inhibiting effective amount of the compound of claim 1.

14-20. (canceled)

21. The method of claim 4, wherein the patient is selected from the group consisting of those patients who have had a stroke, who are at risk of having a stroke, and who are undergoing neurosurgery.

22. The method of claim 4, wherein the patient is suffering from a traumatic brain injury.

23. The method of claim 4, which protects the neurological organs of the patient from ischemia-reperfusion injury.

24. The method of claim 4, which protects the brain of the patient from ischemia-reperfusion injury.

25. A method for protecting neurological organs of a patient with a neurodegenerative disease in which reactive oxygen species (ROS) are involved, comprising administering an effective amount of the compound of claim 1 to the patient.

26. The method of claim 25, wherein the neurological organ is the brain.
